(19) United States
(12) Patent Application Publication
(10) Pub. No.: US 2005/0101995 A1
(43) Pub. Date: May 12, 2005

(54) Arrangement for receiving electrical signals from living cells and for the selective transmission of electrical stimulation to living cells

(75) Inventor: Emmerich Bertagnolli, Wien (AT)

Correspondence Address: MERCHANT & GOULD PC, P.O. BOX 2903, MINNEAPOLIS, MN, 55402-0903, US

(73) Assignee: INNOVATIONSAGENTUR GESELLSCHAFT, Wien (AT)

(21) Appl. No.: 10/922,449

(22) Filed: Aug. 19, 2004

Related U.S. Application Data
(63) Continuation of application No. PCT/AT03/00055, filed on Feb. 19, 2003.

(30) Foreign Application Priority Data
Feb. 19, 2002 (AT) A 251/2002

(51) Int. Cl. A61N 1/00

(52) U.S. Cl. 607/2

(57) ABSTRACT

The invention involves an array to couple a live cell, in particular a nerve cell, with an electronic circuit to pick up directly or indirectly electrically active cell signals and/or to electronically stimulate the cell, with a passive electronic element (C1, R1), where one terminal is connected to a reference potential (M), and the other terminal is connected to a switched output of an electronic switch (T1) the input of which can be connected to a signal or voltage source (point B), where an electrically conducting contact element (1) can be brought into contact with the cell (2) and is connected between the output of the electric switch (point P) and the associated terminal of the passive electronic component (C1, R1).

The invention involves an array to couple a live cell, in particular a nerve cell, with an electronic circuit to pick up directly or indirectly electrically active cell signals and/or to electronically stimulate the cell.

Below an interaction of the coupling array with a cell where general, directly or indirectly electrically active cell signals, and especially electric signals emanated by nerve cells are picked up and processed is described as “passive mode”. An interaction of the coupling array with a cell that causes the cell to be electrically stimulated is described as “active mode”.

Without limiting the generality of the inventive approach, the invention presented may also be implemented at a large scale, i.e. on the basis of existing integration processes of micro- and nanoelectronics, where initially only such processes and materials are used that are, on the one hand, compatible with a modern manufacturing environment for silicon chips and, on the other hand, not biologically harmful. In a further development, other electronic structures that are capable of data processing, such as, i.a., organic circuits, are conceivable and feasible.

Previous processes use, on the one hand, optical methods based on the voltage dependence of colorants (e.g. fluorescence) or extra-cellular metal electrodes. On the other hand, they use invasive methods, e.g. electrodes inserted directly in the cell to detect intracellular potentials. Among the category of invasive electrodes is the silicon needle provided with photolithographically produced circuits and contacts on the tip.

The reverse function, electric stimulation of a cell, has so far been similarly performed by inserted electrodes.

In addition to the technical solutions mentioned to detect and stimulate single cells, there are the so-called cuff electrodes. This is a cuff covered by a wire net and placed around a nerve fibre (including supply and support tissue). However, cuff electrodes allow only a summary pickup and impression of potentials.

Considering that the invasive access to the cell is concomitant to an injury to or direct/indirect destruction of the cell, there is a need for high spatial and temporal resolution and electric sensitivity for non-invasive (or non-penetrating) access to the cell. This requirement is not met by any of the existing devices or methods.

Previous non-invasive solutions based on MOS transistors are inherently based on influencing the inversion layer in the channel of a MOSFET, either directly by placing the nerve cell or axon on the gate dielectric, or indirectly by placing this electrically effective cell part on the gate.

In the one case, the contact between cell and transistor is made so that part of the cell membrane is placed on or made to contact the transistor in the channel. This area is separated from the substrate by the gate dielectric, usually a natural oxide or an artificially grown oxide. Charges or changes in charges of the contacting cell or cell membrane affect the channel conductivity of the transistor. The coupling strength so far achieved with this method is in the range of a 10% change in the gate to cell membrane potential. The MOS transistor, screened off by oxide and with the exception of the leads sticking out on the sides, is completely immersed in electrolyte liquid that is enriched with a nutrient solution, which is extremely detrimental to its life (metal ions, and especially Na and K ions migrate to the gate dielectric, causing progressive degradation). In addition, partially formed channels of variable or weak coupling will be formed, not least due to the incomplete screening of the channel area.

For such reasons, this approach offers only a limited solution for a few single interfaces between the tissue and the electronic system. In view of the lack of regular wiring options for the transition, this solution allows only a limited number of scanning cycles, and on-site amplification and processing of signals is subject to difficult marginal conditions.

Furthermore, this system precludes any direct actuator stimulation.

Another known approach is based on the coupling of a neuron to an MOS gate based on a direct metal galvanic contact between the cell membrane and transistor gate. Here again, the measuring principle is based on the influence on the MOSFET channel exerted by the charge of the gate. Here, an advantage is enjoyed in that the sensitive gate oxide need not be exposed directly to the nutrient solution so that this system can be expected to have a longer useful life.

In both cases, the selection principle is based on the use of differential amplifiers (generally of the CMOS type) to detect changes in conductivity of the contact transistor compared to a non-contacted regular transistor.

In order to stimulate the cell, separate stimulation circuits and suitable contact configurations are required in both cases, e.g. by running ring contacts around the contact transistor.

The present invention seeks to find a solution for the disadvantages of the current state of the art as described above.

In order to meet this object, an array is provided to couple a live cell, in particular a nerve cell, with an electronic circuit to pick up directly or indirectly electrically active cell signals and/or to electrically stimulate the cell in accordance with the characterising features of claim 1. Beneficial developments of the invention are described in the subclaims.

In contrast to all previously known approaches, this coupling array according to the invention allows both the solely passive picking-up of directly or indirectly electrically active cell signals as well as the direct active electric stimulation and influencing (activation) of the cell. The selection of passive or active operating mode is made by an external switch and the choice of voltages and currents applied.

The invention may be applied in particular to the following subject areas:
Studies of directly or indirectly electrically active activities of cells and mechanisms of propagating the impulse of action potentials along axons (nerve fibres).
Signal processing in networks of live neurons.
Simultaneous detection, listed by place and time, of the response to signals/stimulation by a large number of cells/neurons and the associated study of electric wiring of cells/neurons.
Building or assembling of biosensors, in particular neuronal biosensor or sensor/actuator systems and arrays.
Implementation of neuron-electronic circuits.

Other possible applications of the invention concern the design of sensoric and sensomotoric actuators and receptors for prostheses and the implementation of electronic substitutes for nerve fibres, especially for damaged or cut nerve fibres.

In addition, the invention offers an opportunity to study the effect of chemical and physical stimuli, and especially of medicinal drugs and bioactive media on the function and functioning of cells, tissues and tissue parts.

The basic idea of the invention is based on contacting a live cell with passive electric components such that any change in the electric properties of the cell or part thereof can be electrically picked up by a selection circuit and that this circuit also allows to apply electric signals or stimuli to this cell or part thereof.

In a preferred development of the coupling array according to the invention, the electronic switch is designed as a switching transistor, preferentially a field effect transistor. This ensures excellent switching behaviour at minimum power consumption.

In the long term, biological cells can survive only when they are kept in a liquid environment, generally in a so-called nutrient solution. Accordingly, the structure of the circuit according to the invention should allow the biological cell to be placed in a liquid. According to the invention this is ensured by the provision of a container for the nutrient solution into which container at least one contact element projects or constitutes at least part of the inside. To give due regard to the importance of this development, embodiments of the invention are described below, where the cell is schematically shown in a liquid container. However, the invention is not limited to this type of embodiment.

In order to ensure a defined measuring potential, a development of the invention provides for an electrically conducting reference electrode connected to a reference voltage, which electrode projects into the interior of the nutrient solution container.

A long useful life of the coupling array according to the invention and minimal influence on the cells thus coupled may be achieved when the electrically conducting contact element comprises a material of low biological effect, preferentially chosen from refractory metals such as platinum, iridium, osmium, tungsten or gold or alloys thereof; or from semiconductor silicides such as platinum silicide, tungsten silicide, titanium silicide; or from a doped monocrystalline or polycrystalline semiconductor such as conductive polysilicon; or from conductive synthetics.

Notwithstanding the simplicity of the embodiments schematically shown, the coupling array according to the invention is limited neither to single cells nor to cells kept in a liquid.

Rather, the array is excellently suitable to electrically couple entire cell clusters, cell unions, or even entire functional cell units or combinations thereof. In such case, contact fields rather than single contacts are arrayed in the liquid container which are each connected and linked electrically according to the invention.

With regard to storing cells, cell clusters, functional cell groups or combinations thereof in a liquid/nutrient solution it must be noted that any aqueous environment, including specifically body or tissue liquids, suffices to achieve an electrolytic coupling.

In a useful development of the invention, a range of coupling arrays is arranged in the form of a matrix of lines and columns, where the input of each electronic switch is connected to a column address circuit and a controlling connection of the electronic switch is connected to a line address circuit.

Furthermore an addressing circuit may be provided for single or group application of supply or signal voltages on column address circuits and of control voltages on line address circuits. Thus, if the matrix shows a structure in the form of i lines and j columns, the function of the i/jth element allows a unequivocal allocation on the cell field assigned to the matrix. If a number of cells, e.g. nerve cells, is brought into contact with such a matrix, then the cells can be contacted individually and signals can be exchanged in the passive or active mode. Due to the particularity of the accepting circuit, i.e. the matrix form, the electric signals can—as described above—be allocated locally. With this, it is possible to determine and contact individual active cells of a more or less large number of cells which may, e.g. constitute a functional union. Such a contact may be either solely in the passive mode, or in the active mode, or, optionally, in both modes alternatively.

If a larger-scale cell ensemble is placed on one or more such matrix-shaped cell fields of electronic single cells, it is possible by activating electronic retrieval (passive/active mode) to determine which type of contact is present on which site, what is the spatial configuration of such contacts and how a signal sequence corresponding to a given application purpose or an algorithm is to be applied.

In this manner, non-active or insufficiently active or non-contacted cells may be similarly determined and may, e.g., be excluded from further interactive processing (passive and/or active mode), through the address circuit having address evaluation tools to detect dysfunctional cells and faulty contacts between the contact element and the cell, where in the case of such detection, if need be, further interaction of such cells or contact elements may be selectively interrupted. For the selective interruption of cell interaction, interruption tools, such as electronic switches or fuses, may be provided.

The coupling array according to the invention permits arranging a large number of coupling arrays on a chip, where the chip is preferentially made of Si-planar technology and may be integrated with other technologies, such as circuits for local amplification, on-chip logic or systems on chips (SoC).

The component density achievable today in accordance with the state of the art permits the design of very large arrays of cell sensors and actuators, where local electronic circuits may optionally be accommodated at any node of such an array. This may be necessary in the case of low signal intensity (e.g. the lift of a cell membrane voltage on activating a nerve cell typically is <60 mV) or if noise sources or noise levels occur.

Using this sensor array and suitable address circuits, it is possible to observe/measure the signal propagation and processing in biological cells, cell clusters and functional cell systems, particularly in nerve cell tissues, and if necessary data thus obtained may be further processed.

In another development of the invention, the nutrient solution container is arranged on the chip. This allows entire cell clusters, cell unions or even entire cell units or combinations thereof to be stored in liquid within contact range of the contact elements and enables the chips to be used in an aqueous environment, specifically also in body or tissue liquids that permit electrolytic coupling. In this development, the top layer of the chip, the so-called passivation, is to be designed so that the contact points or contact surfaces of the contact elements are attached to special penetration points and these are then brought into contact with the cells.

The coupling array according to the invention can be placed directly in live tissue or openings thereof. For contact with the tissue liquid, the electrolyte, a suitable contact, possibly also integrated, is to be provided according to the invention.

In a development of the coupling array according to the invention, a capacitor is provided as the passive electronic component. Here, the choice of passive and active operating mode is set by an external switch and selection of voltages and currents applied.

In an alternative development of the coupling array according to the invention, an electrical resistor is provided to serve as the passive electronic component. In this case, the cell/resistor system constitutes a voltage divider, where the cell potential can be picked up through the contact element. Reading, writing and refreshing of the potential are possible by a suitable external switch and the application of suitable voltages and currents.

Below, embodiments of the invention are described with reference to the drawings.

However, the invention is not limited to these embodiments.

Of the drawings,

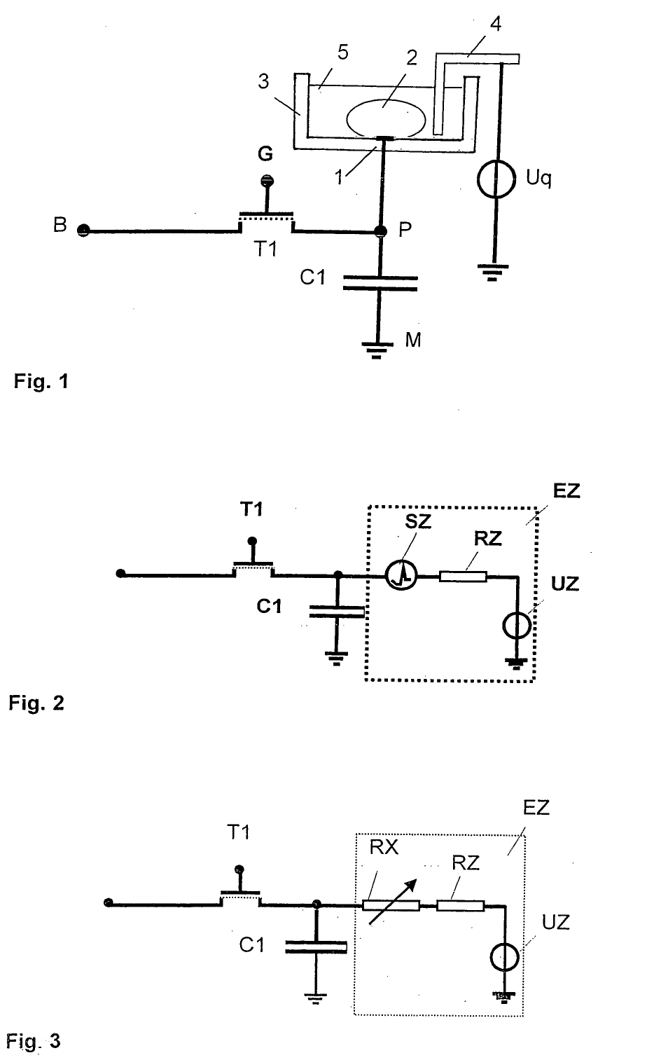

FIG. 1 shows a schematic diagram of a coupling array according to the invention, with a capacity as passive electric coupling element.

FIGS. 2 and 3 show the embodiment of FIG. 1 together with several equivalent circuit diagrams of cells to be coupled.

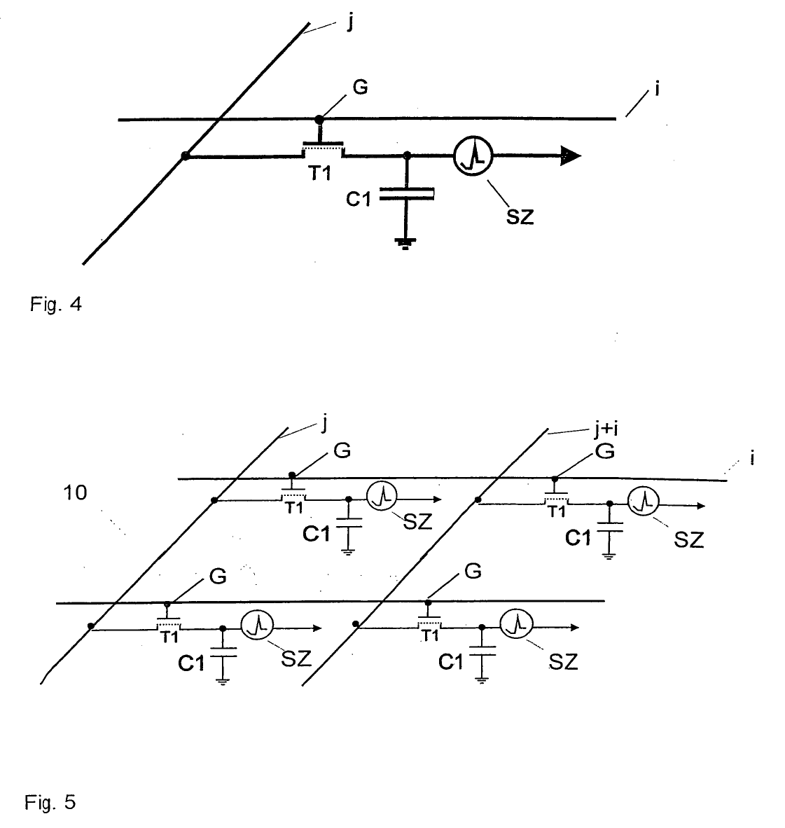

FIGS. 4 to 7 show matrix arrays of the coupling array according to the invention with a capacity as passive electric coupling element.

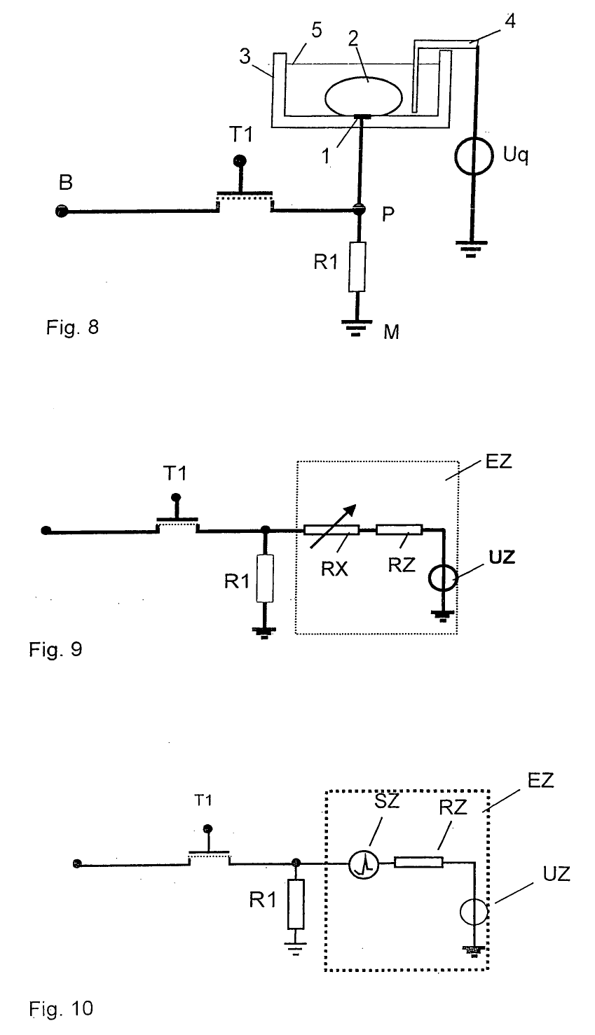

FIG. 8 shows a schematic diagram of a coupling array according to the invention with a resistor as the passive electric coupling element.

FIGS. 9 and 10 show the embodiment of FIG. 8 together with various equivalent circuit diagrams of the cells to be coupled.

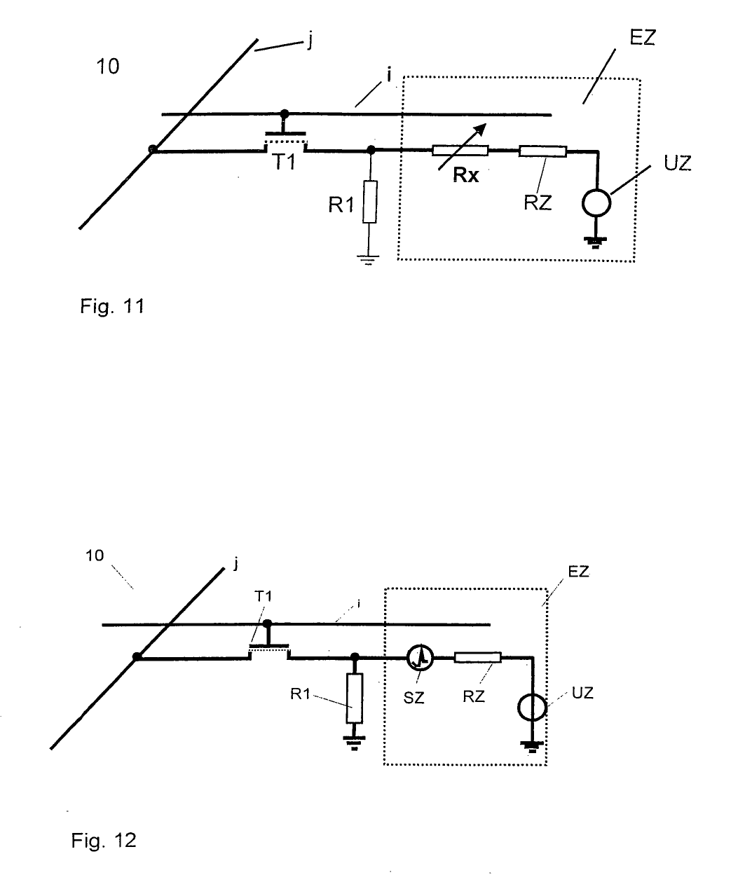

FIGS. 11 and 12 show matrix arrays of the coupling array according to the invention with a resistor as the passive electric coupling element.

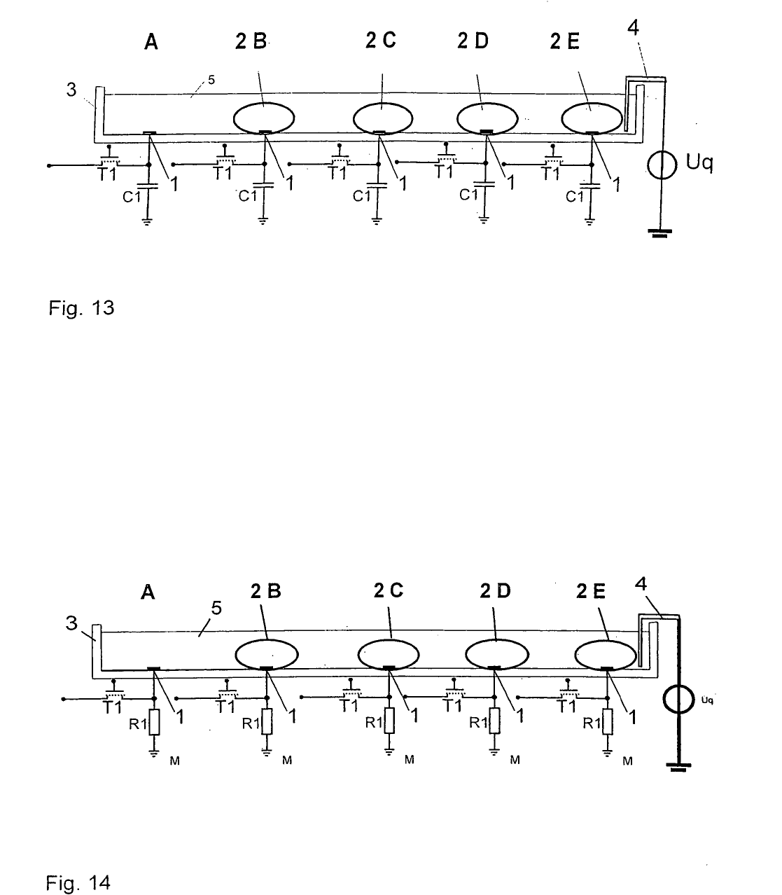

FIGS. 13 and 14 show the coupling of several cells held in a container of nutrient solution to coupling arrays according to the invention.

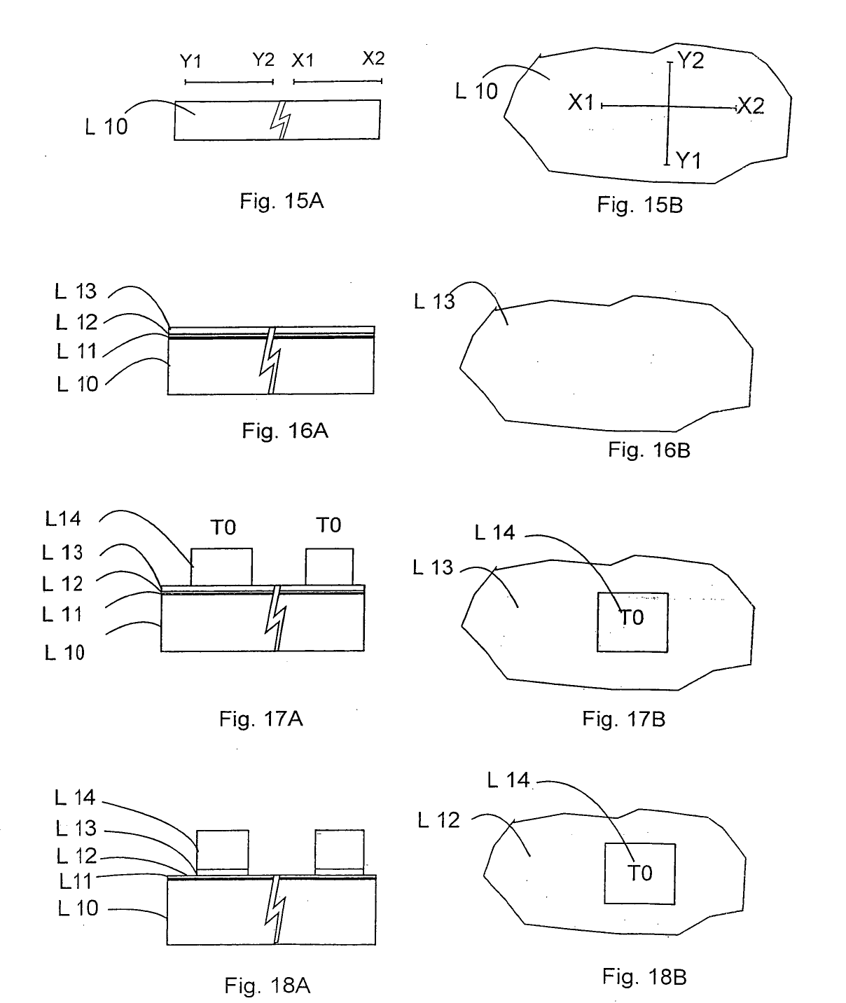

FIGS. 15 to 50 show a process according to the invention to produce a coupling array according to the invention with a capacity as passive electric coupling element.

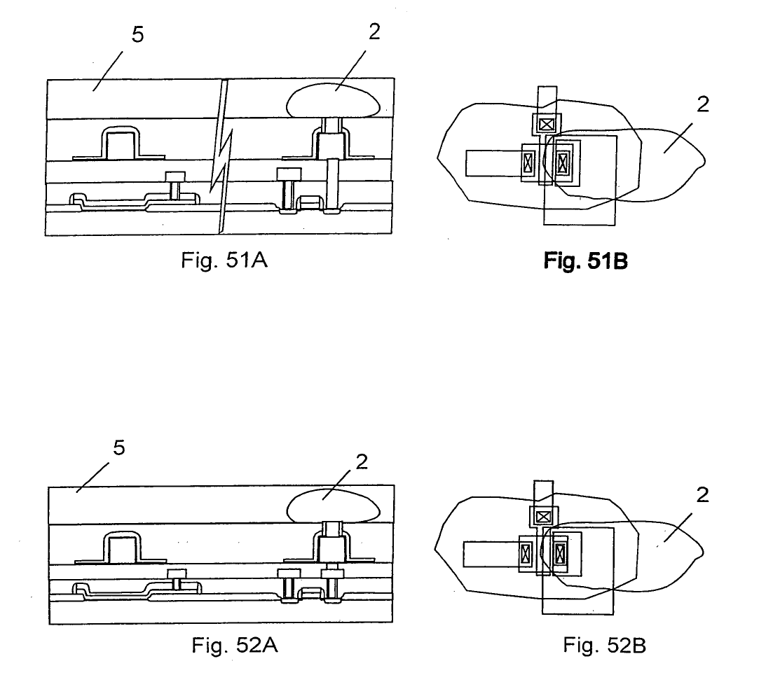

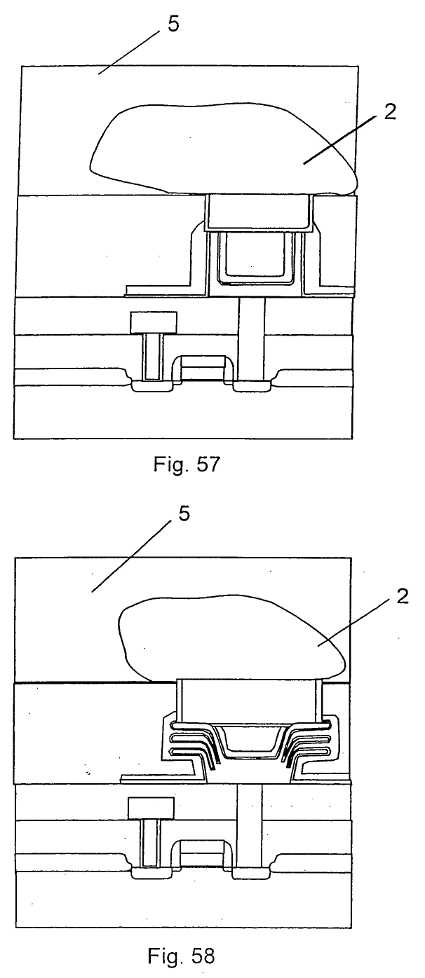

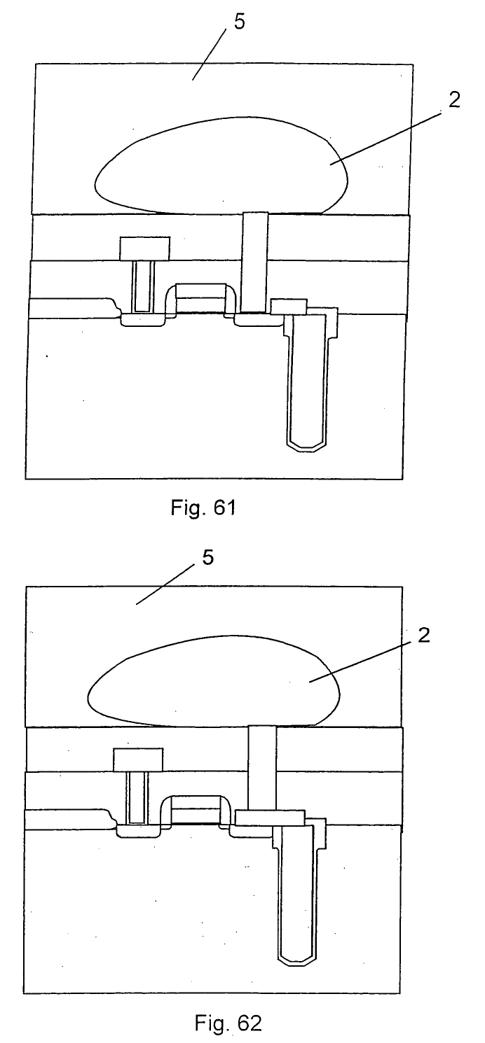

FIGS. 51 to 62 show embodiments with different capacities.

In the description below, identical or similar elements are described with the same reference number so that it will not be necessary to repeat the description of such elements.

FIG. 1 shows a schematic diagram of an array according to the invention for the electric coupling of a biological cell 2 to this array.

The live cell 2 is placed in a nutrient solution 5 which constitutes an electrolyte. Nutrient solution 5 is placed in a nutrient solution container 3. The potential of the nutrient solution 5 is maintained at a defined potential, such as a mass potential or potential Uq vis-à-vis the mass, by a reference electrode 4, e.g. a platinum electrode or hydrogen electrode.

The cell 2 is electrically coupled to an electronic circuit by a direct galvanic contact with a contact element 1 made of a conductive material, e.g. a metal that has a slight or negligible biological effect, such as, e.g., platinum, iridium, osmium or gold, or a doped monocrystalline or polycrystalline semiconductor such as conductive polysilicon; or a conductive synthetic material.

At point P, the contact element 1 is connected to one terminal of capacitor C1 and an electronic switching element T1, also described below as selection transistor. The second terminal of capacitor C1 is applied to a reference potential, or to be more specific, the mass potential M. The selection transistor T1 may be connected to a signal or voltage source through an input terminal at point B. Its switching state (ON/OFF) is controlled through gate G.

Without interpreting this as a restriction of the general approach, the figures indicate the reference potential as a mass potential M. It goes without saying that any other potential or reference voltage such as VDD/2 may be used alternatively without impairing the function of the array.

Below, the functional principle of the array according to the invention is described.

Each change in the membrane potential of cell 2 or the cell part placed on the contact area of contact element 1 is transmitted via contact element 1 to capacitor C1, which is charged accordingly. The branch point P in FIG. 1 changes its potential to reflect changes of cell 2. With the selection transistor T1 being blocked, this change in potential cannot be balanced and no charge (with the exception of unavoidable leak currents) may be discharged. If the blocked election transistor is made conductive by the application of a suitable control voltage at gate G (gate voltage), then the charge can be discharged through point B and the potential/charge is reduced. Conversely, a potential applied to point B can be applied to point P when the selection transistor T1 is made conductive (switched through). This potential is then transferred to cell 2 via contact element 1. With this, cell 2 can be electrically stimulated.

In general, this electronic array constitutes a kind of storage cell that is charged, i.e. “written on” by the action potentials of the cell. Discharging the charge in this context constitutes the “reading” of charges previously imprinted, i.e. the action potentials. If a potential is applied to the cell from outside, this procedure constitutes the stimulation of a cell.

If the cell signal is to be only read without changing it in its time means, it is necessary to return the information thus read out, i.e. the charge discharged for pick-up, promptly to capacitor C1, i.e. the cell information needs to be rewritten/restored.

Depending on the system leakage rate, this procedure may also be necessary to maintain the cell potentials for a sufficient length of time.

FIG. 1 shows a basic setup of the coupling array according to the invention. The live cell is placed in a container 3 holding a nutrient solution 5, where the contact point of contact element 1 is represented schematically on the bottom of the container. This contact element is connected at point P to the selection transistor T1 and capacitor C1. The voltage source Uq is optional and is intended to demonstrate that the electrolyte potential of the nutrient solution 5 may differ from the mass potential or reference potential.

Depending on the effectiveness of the contacts or the signal/stimulation response of the cell, the cell in the equivalent electric circuit diagram EZ may be perceived as a generator Sz that sends signal impulses, as shown in FIG. 2, where the cell shows a cell resistance Rz, or also as a signal/stimulation-dependent variable resistance Rx that acts serially to cell resistance Rz (see FIG. 3). FIG. 3 essentially shows a resistive interaction between the electronic circuit T1, C1 and the biological part, i.e. the cell. In this case, the electrically effective change of the cell is essentially a change in resistance.

FIGS. 4 and 5 provide a schematic diagram of cell selection in a cell matrix in the case of cells that represent the function of a generator Sz for electric impulses. In line with the above, “reading” (i.e. passive mode), “writing” (i.e. active mode) as well as “refreshing” or “holding” the charge are possible, as is restoring the potential after reading.

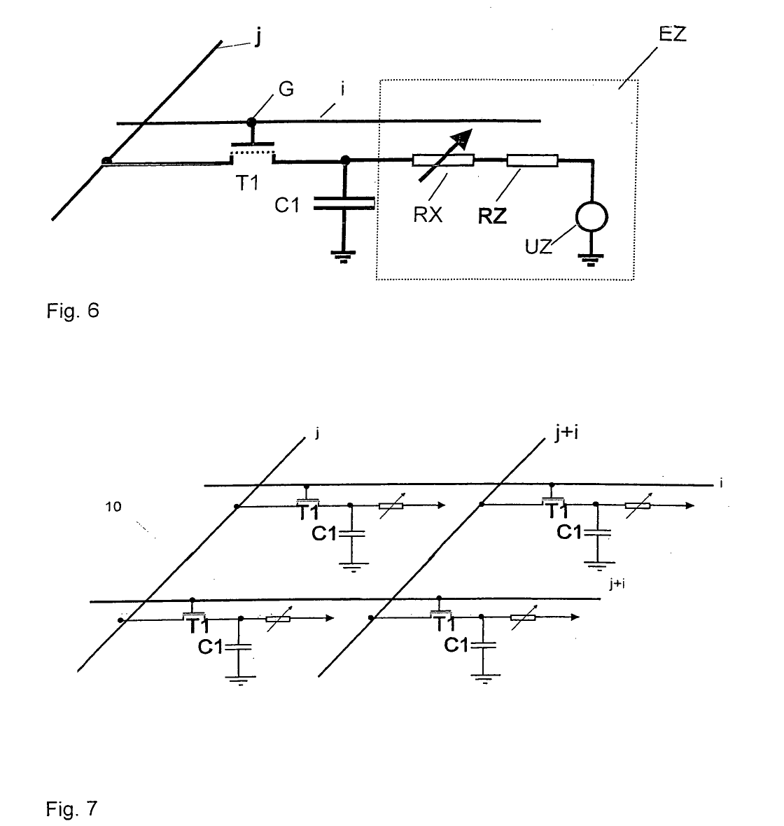

FIGS. 6 and 7 provide a schematic diagram of cell selection in a cell matrix 10 in the case of cells which respond mostly resistively to signals/stimulation. In line with the above, “reading” (i.e. passive mode), “writing” (i.e. active mode) as well as “refreshing” or “holding” the charge are just as possible as is restoring the potential after reading.

FIGS. 8 through 10 show another coupling array according to the invention which, contrary to the previous embodiments includes a resistor R1 rather than a capacitor. In these cases, the cell/resistor system constitutes a voltage divider. The potential at point P can be picked up through the selection transistor T1. As above, reading, writing and refreshing the potential is possible by applying suitable voltages to point B. The requisite matrix arrays of such a coupling in a cell field are shown in FIGS. 11 and 12, where FIG. 11 shows an example of cell selection in the case of a primarily resistive coupling, and FIG. 12 shows an example of cell selection in the case of an active/resistive coupling.

In order to demonstrate the robustness and error tolerance, FIGS. 13 and 14 show the very general case of a coupling array according to the invention in the presence of a larger number of cells of false usage and cell failure.

In the case of an array with coupling capacity C1 (FIG. 13) it can be easily seen that the absence of a cell (see A) on a contact element 1 is easy to detect through the selection transistor T1 (potential at point P is always equal to the potential of the nutrient solution 5, i.e. the electrolyte). For as long as the selection transistor T1 remains switched off, this contact element on place A will remain ineffective.

Similarly, a dysfunctional cell (2C) can be detected by showing no or an inadequate kind of stimulation/response pattern. This cell 2C may be similarly switched off without any problems through the associated selection transistor T1. Cells 2B, 2D and 2E show a satisfactory stimulation/response pattern, i.e. the cells respond actively/passively and thus indicate that the cell contact is fully functioning.

In summary it may be said that the array, through an appropriate addressing circuit and addressing processes (i.e. algorithms), allows excluding faulty contacts (A) and dysfunctional cells (2C) from further interaction without impairing interaction between the remaining cells (2B, 2D, 2E).

The coupling array shown in FIG. 14 with the resistance element R1 as a coupling element is similarly able to detect faulty contacts (at A) and dysfunctional cells (2C).

However, if the cell only responds in a resistive manner, a leak current is to be expected which can be stopped only when the feed is destroyed through a kind of fuse.

As an example, this may be done by shifting the potential of point B jointly with point P, i.e. jointly with the nutrient solution 5 (the electrolyte) sufficiently against potential point M that the circuit is destroyed in the branch section between P and M by flowing current same as occurs in a cut-out fuse. This procedure does not involve any potential difference between points B and P so that the cells in the nutrient solution are not affected.

Without limitations and within the meaning of the array according to the invention, the container 3 holding the nutrient solution 5 and the cells 2B-2E may alternatively be applied directly to a semiconductor chip. In this case, the top layer (the so-called passivation) may be designed so that the contact elements 1 are attached to specific penetration points which are then put into contact with the cells.

Similarly, components with such contacts on the surface may be inserted directly into live tissue. In this case, a suitable, possibly integrated, contact needs to be provided for contacting the tissue liquid (electrolyte). Below an embodiment of the coupling array according to the invention with a capacitative neuron coupling is described.

The procedural steps are shown in FIGS. 1 5A to 50A as a cross-section and in FIGS. 15B to 50B as a so-called layout, where Figure numbers given below without the “A” and “B” indices are joint references to cross-section and layout.

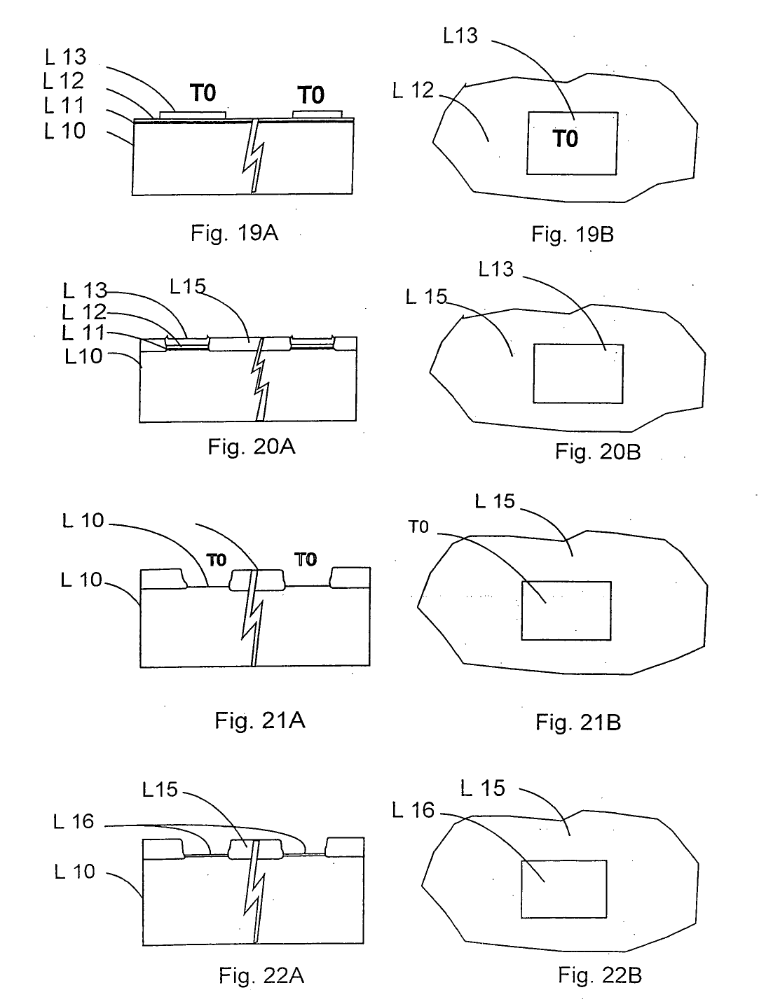

On a substrate (layer 10) (FIG. 15), which will generally be p-doped silicon, three layers are consecutively deposited or generated (FIG. 16): layer 11 (e.g. silicon dioxide), layer 12 (e.g. polysilicon), layer 13 (e.g. silicon nitride). Next, using a photographic technique 1, the transistor T0 is defined across a lacquer mask (layer 14) (FIG. 17) and the area thus defined is created by etching layer 13 (FIG. 18). In the next step, the lacquer mask is removed (FIG. 19) and, as shown in FIG. 20, the area outside T0 is oxidised (LOCOS, local oxidation of silicon), in order to build the so-called field oxide area (layer 15) outside the transistor area T0. The field oxide area (layer 15) may alternatively be built by any other technique such as shallow trench isolation.

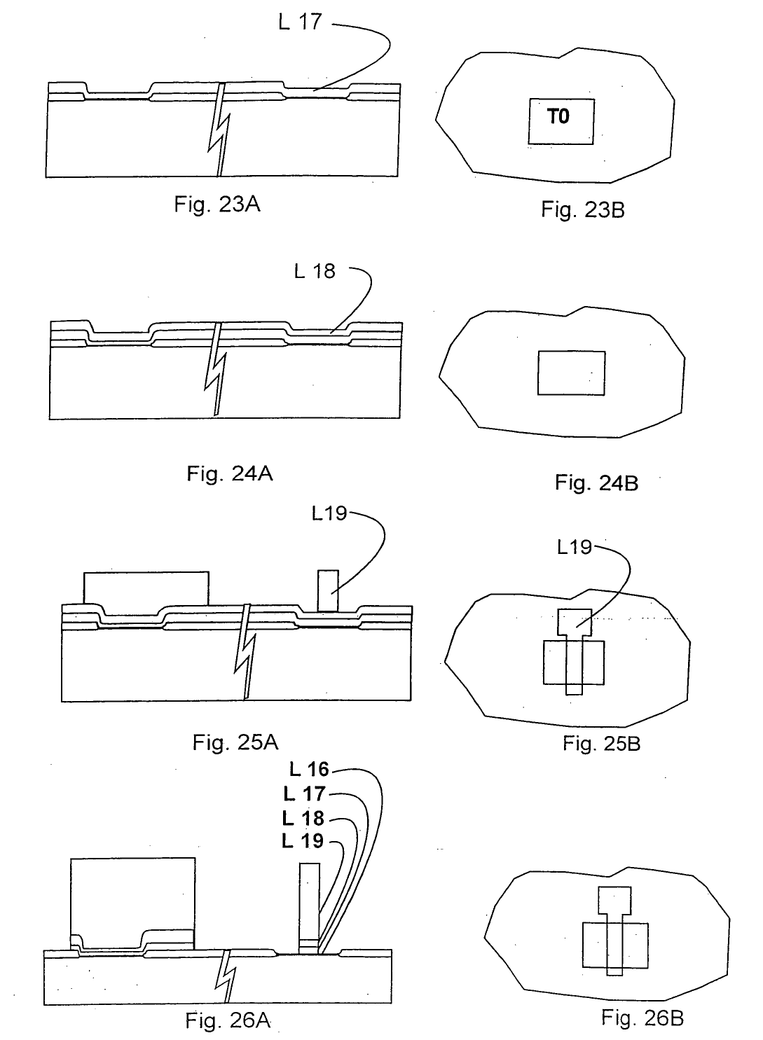

Following oxidation, the remains of layers 13, 12, 11 are removed (FIG. 21). If necessary, etching of a suitable kind is used to remove the residual oxide (e.g. native oxide), to expose the transistor area and build up the gate stack: after growing the gate oxide (layer 16) (FIG. 22) or another suitable dielectric, a polysilicon layer (layer 17) (FIG. 23) is either deposited in a doped condition or deposited without doping and subsequently doped (e.g. n-doped). According to the state of the art, this layer may also be made of another conductive material, e.g. a metal (metal gate technique).

Next, a thin silicon dioxide layer or other suitable dielectric (layer 18) is grown or deposited on this polysilicon layer (FIG. 24).

In the next step, gate G0 and the gate level are defined by photolithographic means (layer 19), and built by structurising layers 18, 17 and 16. With this procedure the gate G0 (layer 17) is produced (see FIGS. 25 and 26).

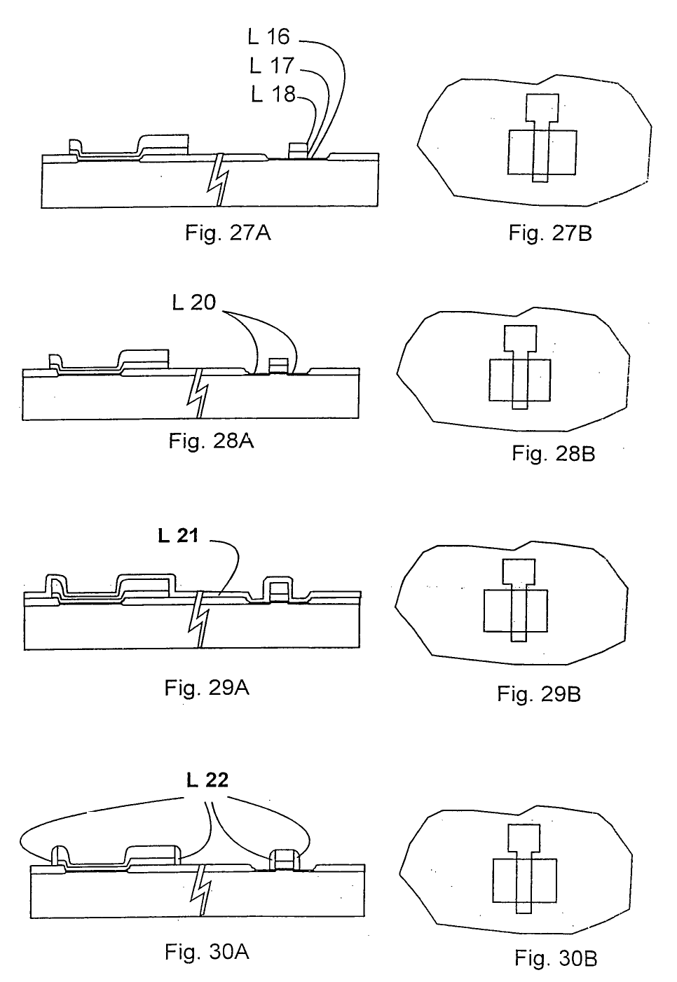

Next, the photolacquer (layer 19) is removed (FIG. 27) and, through an implantation process or other suitable doping process, the so-called lightly doped drain zone (layer 20) is produced self-adjusted (FIG. 28).

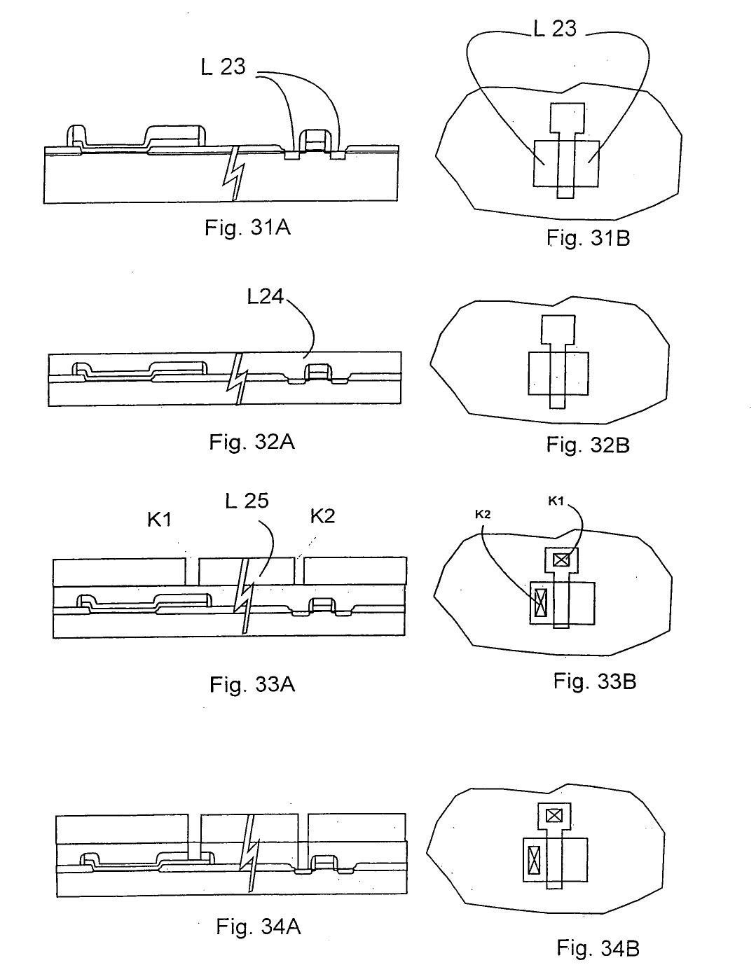

In the next step, through conformous deposition of a dielectric (layer 21) (FIG. 29), followed by anisotropic etching-back, a so-called sidewall spacer (layer 22) is built (FIG. 30). Once the spacer has been built, the so-called heavily doped drain (HDD) implantation is performed (layer 23) (FIG. 31) and the source/drain connection area defined. In the embodiment shown, this HDD implantation is followed by the deposition of a dielectric layer, e.g. silicon dioxide (layer 24) (FIG. 32), which is deposited and levelled or deposited for levelling.

Next the contact windows K1 and K2 are defined by a photographic technique 3 (layer 25) (FIG. 33) and made by etching layers 24, 18 and, if necessary, 16 (FIG. 34).

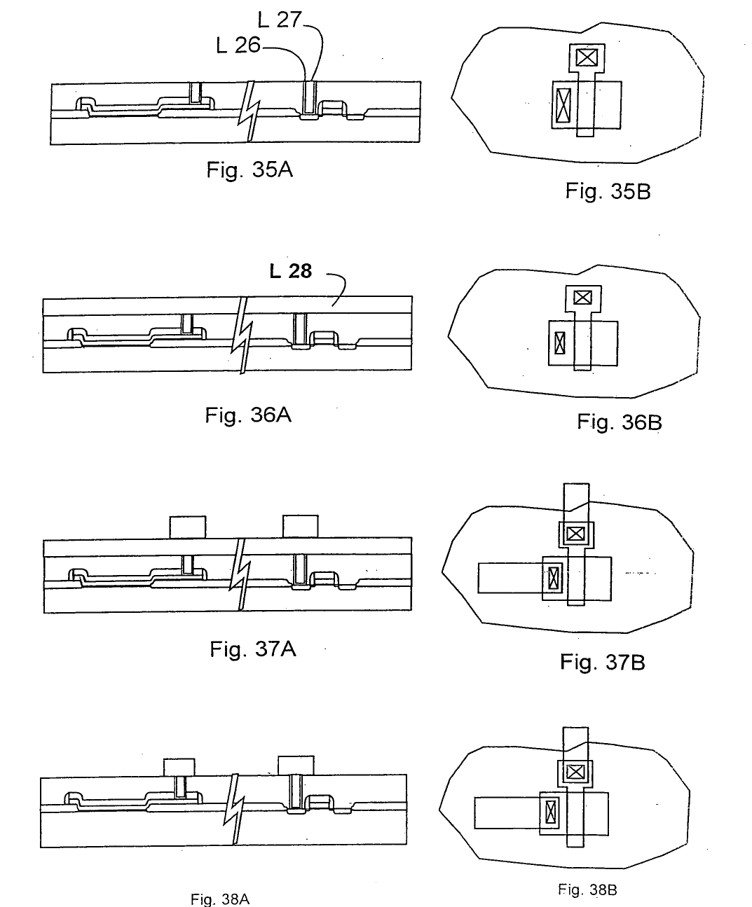

If necessary a barrier layer (Layer 26) is used to fill the contact holes with a conductive material (e.g. tungsten/titanium nitride) (layer 27) (FIG. 35).

As the next step, a metallic layer (layer 28) is applied (FIG. 36) and structured using a photographic technique 4 (FIGS. 37 and 38).

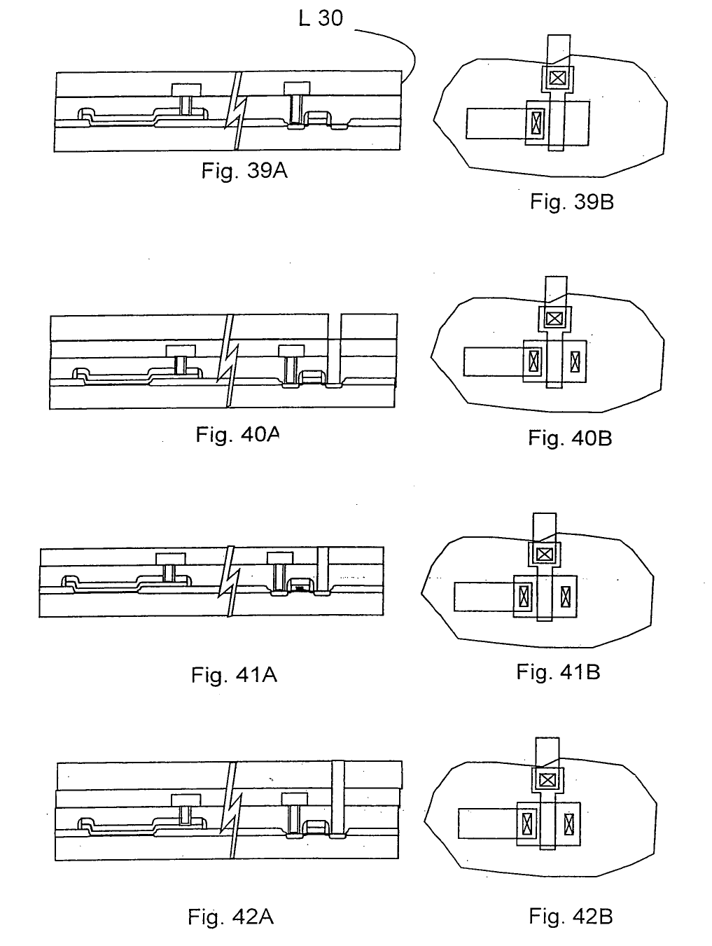

Upon completion of the metallisation layer M1 (layer 28), a dielectric layer is applied to the embodiment, which is either levelled or deposited for levelling (layer 30 in the figure) (FIG. 39).

Next, a photographic technique 5 is used to define the contact hole KC (layer 28) (FIG. 4), and openings are produced by etching the respective layers (layers 30 and 24, if necessary 16), which openings reach to the terminal zone of the transistor (FIG. 41).

This contact hole KC is then filled with a conductive material (e.g. doped polysilicon doped, or a metal such as tungsten/titanium nitride) (layer 32) (FIG. 42).

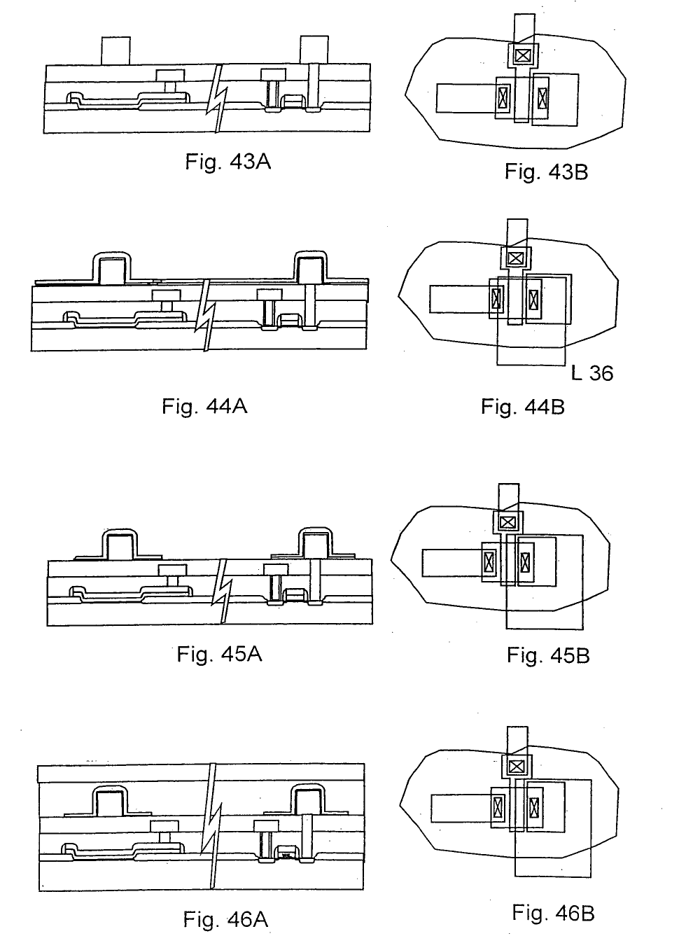

In the next step, a conductive layer (layer 33) is applied and structured using a photographic technique 6 (building the capacitance electrode) (FIG. 43).

Next a thin dielectric is deposited (layer 35), on which a conductive layer (layer 36), e.g. doped polysilicon, is deposited (FIG. 44) which acts as the second electrode (counter-electrode) for the capacitor structure.

Using a photographic technique 7, the layout of this second electrode of the capacitor structure is defined and built by etching the layer (FIG. 45).

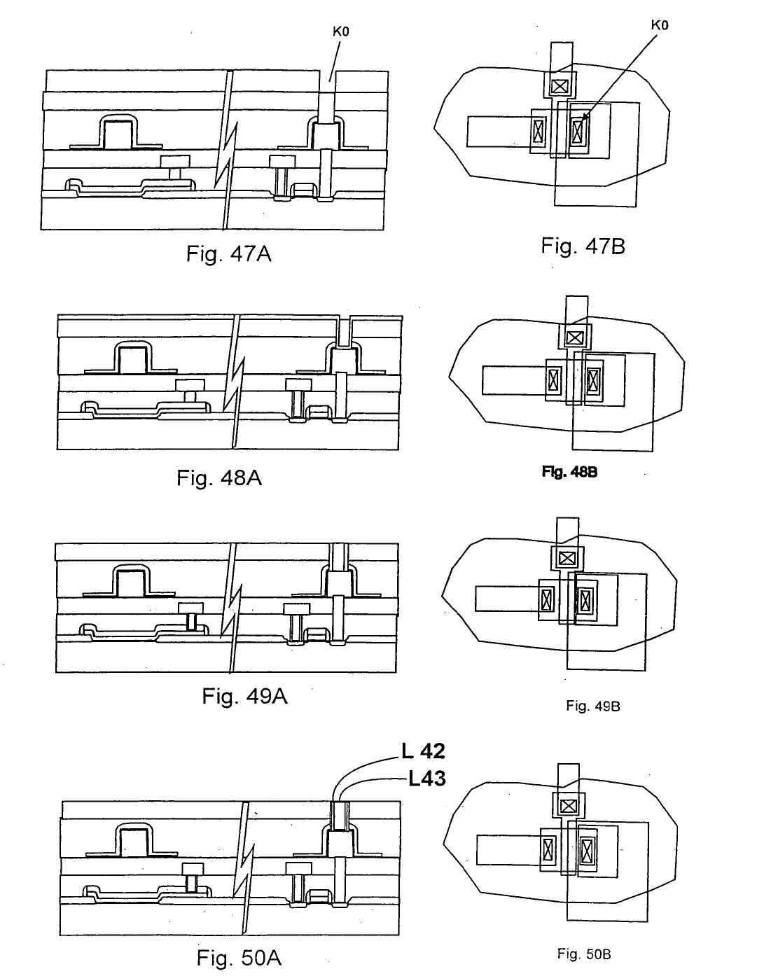

Upon removing the lacquer (layer 37), a thick dielectric single or multiple layer is deposited and if necessary levelled or deposited for levelling (layers 38 and 39) (FIG. 46).

Next, the contact hole KO is defined by a photographic technique 8 (layer 40), and an opening is made by etching the respective layers (layers 39 and 38, and layers 36 and 35), which opening stretches to the first electrode of the capacitor structure (FIG. 47). Upon etching, the photographic lacquer is removed.

Upon depositing a conformous dielectric (layer 41) (FIG. 48) and by anisotrope back-etching within the meaning of the spacer technology (FIG. 49), this contact hole is lined inside with an insulator while the contact to the first electrode of the capacitor structure remains in place.

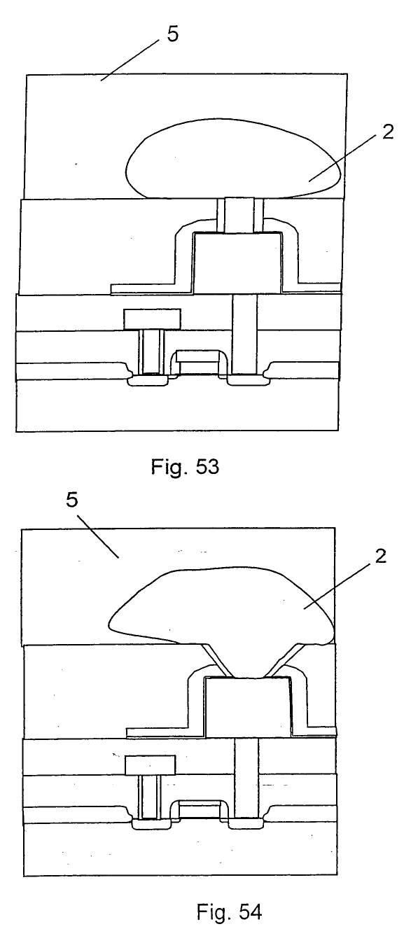

If necessary using a barrier or adhesive barrier, this contact hole is filled with a conductive material (e.g. doped polysilicon doped or a metal such as tungsten/titanium nitride) (layers 42 and 43) (FIG. 50) so that the surface will remain essentially level. This contact area provides for the coupling to the biological cell according to the invention. As shown in FIG. 51A (cross-section) and 51B (layout) and in the enlargement of FIG. 53, the cell contact can be made directly through this docking point. The figure described here will be perceived as Version A.

Without limiting the generality of the invention, in a Version B (FIGS. 52A (cross-section) and 52B (layout)) the first electrode of the capacitor structure may not reach directly to the terminal area of the transistor, but only to a metal contact which is in turn connected to the transistor through a normal contact.

In a Version C (FIG. 54) of the invention, the cell contact to the capacitor can be made without using a metallic interface. In this embodiment the dielectric layer to which the cell attaches, is of a conical shape.

In a Version D (FIG. 55), the cylinder-shaped capacitor is replaced by a hollow cylinder-shaped capacitator.

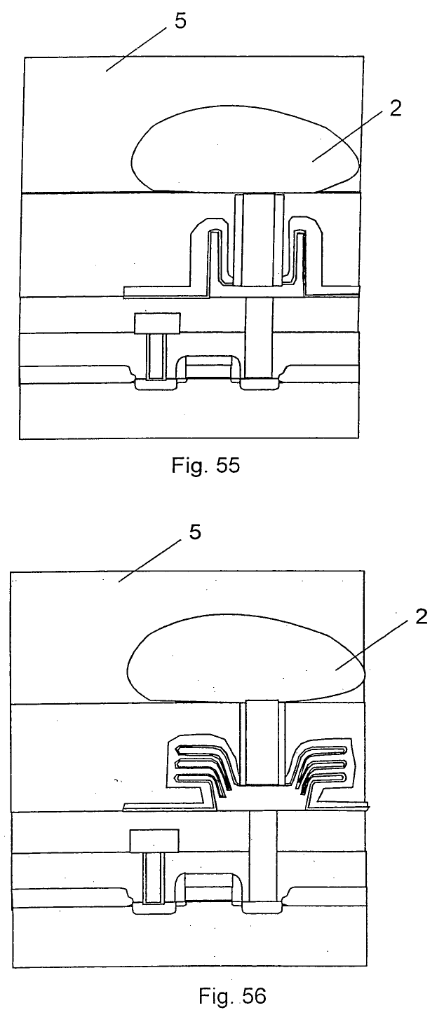

Similarly, in a Version E capacitor structures are conceivable which show a calyx-like structure up to surface enlargement (FIGS. 56 through 58).

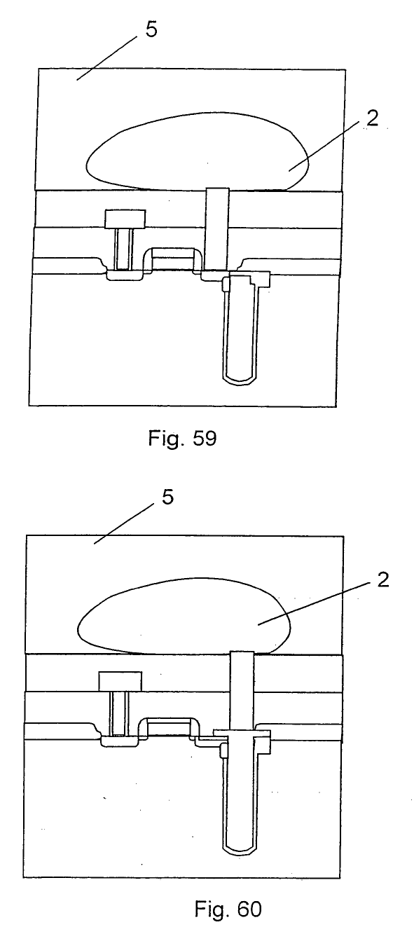

Furthermore, in a Version F capacitors are conceivable that are shaped as trench cells (FIGS. 59 through 62). In this case, the cells may be connected directly to the internal electrode or to the terminal zones. Alternatively, of course, a regular metal contact may be provided to which the cells are connected.

1. Array to couple a live cell, in particular a nerve cell, to an electronic circuit to pick up directly or indirectly electrically effective cell signals and/or electrically stimulate the cell, wherein a passive electronic element (C1, R1) that is connected with one terminal to a reference potential (M) and with another terminal to a switched output of an electronic switch (T1) the input of which can be connected to a signal or voltage source (point B), where an electrically conducting contact element (1) can be attached to the cell (2) and is connected between the output of the electric switch (point P) and the terminal of the associated passive electronic component (C1, R1).

2. Coupling array as claimed in claim 1, wherein the electronic switch (T1) is a switching transistor, preferentially a field-effect transistor.

3. Coupling array as claimed in claims 1, wherein a container to hold a nutrient solution (3) is provided, where at least one contact element (1) either projects into the container or at least partly forms this container.

4. Coupling array as claimed in claim 3, wherein an electrically conducting reference electrode (4) is provided connected to a reference potential or reference voltage (Uq), where the reference electrode projects into the interior of the container holding the nutrient solution (3).

5. Coupling array as claimed in claim 1, wherein the electrically conductive contact element (1) is made of a material of low biological effect, preferentially chosen from refractory metals, such as platinum, iridium, osmium, tungsten or gold, or alloys thereof; or of semiconductor silicides, such as platinum silicide, tungsten silicide, titanium silicide; or of a doped monocrystalline or polycrystalline semiconductor, such as conductive polysilicon; or of conductive plastics.

6. Coupling array as claimed in claim 1, where a large number of coupling arrays are arrayed in the form of a matrix (10) of lines (i, i+1) and columns (i, j+1), wherein the input of each electronic switch (T1) is connected to a column address circuit (i, j+1) and a control terminal (G) of the electronic switch (T1) is connected to a line address circuit (i, i+1).

7. Coupling array as claimed in claim 6, wherein an address circuit to apply supply or signal voltages to column address circuits (i, j+1) singly or in groups and to apply control voltages to line address circuits (i, i+1).

8. Coupling array as claimed in claim 7, wherein the address circuit has address evaluation tools to detect dysfunctional cells (2C) and faulty contacts (A) between the contact element and the cell, where in the case of such detection further interaction between these cells and contact elements can be selectively interrupted.

9. Coupling array as claimed in claim 8, wherein interrupting tools such as an electronic switch or a cut-out fuse are provided for the selective interruption of interaction between cells.

10. Coupling array as claimed in claim 1, wherein a multiple number of coupling arrays are arrayed on a chip, where the chip is preferentially produced by the Si-planar process and may be integrated with other technologies, such as circuits for local amplification, on-chip logic or systems on chips (SoC).

11. Coupling array as claimed in claim 10, wherein a container to hold a nutrient solution (3) is provided, where at least one contact element (1) either projects into the container or at least partly forms this container, characterised in that and wherein the container holding the nutrient solution is placed in the chip.

12. Coupling array as claimed in claim 1, wherein the passive electronic component is a capacitor (C1).

13. Coupling array as claimed in claim 1, wherein the passive electronic component is an electric resistor (R1).
